(19) United States
(12) Patent Application Publication
(10) Pub. No.: US 2024/0082146 A1
(43) Pub. Date: Mar. 14, 2024

(54) COMPOSITIONS AND METHODS OF IMPROVING THE SKIN MICROBIOME

(71) Applicant: SEED HEALTH INC., Venice, CA (US)

(72) Inventors: Rajat Dhir, Venice, CA (US); Anthony L. Almada, Venice, CA (US); Azza A. Gadir, Venice, CA (US)

(21) Appl. No.: 18/518,528

(22) Filed: Nov. 23, 2023

Related U.S. Application Data
(63) Continuation of application No. 17/089,469, filed on Nov. 4, 2020.
(60) Provisional application No. 62/930,266, filed on Nov. 4, 2019.

(51) Int. Cl. A61K 8/99; A61K 8/34; A61K 8/49; A61K 8/64; A61K 8/9789; A61K 31/05; A61K 31/353; A61K 31/436; A61K 35/741; A61K 36/185; A61K 38/16; A61Q 19/00

(52) U.S. Cl. CPC A61K 8/99; A61K 8/347; A61K 8/498; A61K 8/64; A61K 8/9789; A61K 31/05; A61K 31/353; A61K 31/436; A61K 35/741; A61K 36/185; A61K 38/164; A61Q 19/00

(57) ABSTRACT

The present invention comprises compositions, systems, and methods of improving the skin and the skin microbiome using bacterial strains and their derivatives, and prebiotics. A useful composition of the invention comprises at least one of (a) a prebiotic that selectively promotes the growth of at least one desirable strain of Cutibacterium acnes (C. acnes); (b) a probiotic comprising at least one desirable strain of C. acnes; and (c) a post-biotic comprising at least one molecule produced by C. acnes.

### CROSS-REFERENCE TO RELATED APPLICATION

This application is a continuation of U.S. patent application Ser. No. 17/089,469, filed 4 Nov. 2020, which claims the benefit of priority under 35 U.S.C. § 119(e) to U.S. provisional patent application 62/890,827, filed 4 Nov. 2019, the entireties of both of which are incorporated herein by reference.

### FIELD OF THE INVENTION

This invention relates generally to improvement of the skin microbiome, and more particularly to compositions and methods for modifying the relative abundance of desirable and/or undesirable strains of Cutibacterium acnes in the skin microbiome.

### BACKGROUND OF THE INVENTION

Acne vulgaris, or simply acne, is a chronic inflammatory skin condition classified by the Global Burden of Disease Study as the eighth most prevalent disease worldwide. The pathophysiology of the condition has been extensively studied, with an increase in sebum production, abnormal keratinization of the pilosebaceous follicle, and an inflammatory immune response all being implicated in its etiology.

Acne vulgaris affects more than 85% of teenagers and more than 10% of adults and has recently been redefined as a complex chronic disease associated with Cutibacterium acnes (C. acnes) (formerly referred to as Propionibacterium acnes (P. acnes)), which takes advantage of an immune-susceptible host. C. acnes colonizes and grows optimally in the presence of lipids and in the absence of oxygen. In addition, sebum secretion, which is intensified during puberty and other hormonally active periods (e.g. the premenstrual period and pregnancy), provides an ideal environment for C. acnes and attracts the bacteria into the hair follicles, which are located deeper within the skin.

In the past, the pathogenic role of C. acnes has been debated because the bacterium is found both on the skin of acne patients and on the skin of healthy individuals. The theory that C. acnes does not cause acne per se, but rather contributes to inflammation once the acne lesion has been formed, has been challenged by the latest epidemiological studies using highly sensitive, improved methods for genetic profiling. Particularly, recent studies have shown significant differences between the C. acnes strains isolated from patients with severe acne compared to healthy controls and striking functional differences among strains from different phylogenetic clusters.

Acne vulgaris continues to burden every generation, and despite the multitude of treatments and products on the market, no single treatment exists that can guarantee a long-term benefit to all acne patients. Antibiotics are most often used as a frontline treatment, but cannot be given long-term, and widespread use of antibiotics is thought to be a factor in the rise of antibiotic-resistant bacteria and may therefore lead to greater harm than benefit. Vitamin A derivatives (isotretinoins) are a second widely used treatment option, as orally administered isotretinoin suppresses sebaceous gland activity and indirectly reduces inflammatory lesions, but isotretinoin, as well as other common oral medications such as oral contraceptives and spironolactone, are not curative drugs, and discontinuation of the treatment is frequently followed, in the absence of appropriate maintenance treatment, by recurrence. Further, due to potentially serious side effects (birth defects, vision problems, mental health problems, hair loss, etc.), isotretinoin is not routinely prescribed to patients. Therefore, acne patients would welcome an alternative, safer treatment that is effective over the long term.

While the chronic inflammatory condition caused by C. acnes is generally considered non-pathogenic, there is a growing body of evidence that points to the bacterium as being a low-virulence pathogen in several types of postoperative infections and other chronic conditions. C. acnes has been associated with endocarditis of prosthetic and native aortic valves, corneal infections, and postoperative endophthalmitis. It has also been recognized as a source of infection in focal intracranial infections and various cerebrospinal fluid shunt infections. Further, a recent study from Japan has shown that C. acnes DNA can be detected in lymph nodes of Japanese individuals with sarcoidosis, a granulomatous disease that results in the inflammation of the lymph nodes, lungs, eyes, liver, and other tissues. C. acnes has been isolated from intervertebral disc material of patients with severe sciatica and it has been hypothesized that low-virulence organisms such as C. acnes can gain access to an injured spinal disc and initiate chronic inflammation. C. acnes has also been isolated from several orthopedic infections, silicone breast prostheses, and prosthetic joint infections, which have been shown to contain bacterial biofilms of C. acnes and/or Staphylococcus epidermidis.

Thus, there is a need for better control of C. acnes on skin surfaces. The present invention addresses this need.

### SUMMARY OF THE INVENTION

The present disclosure provides compositions for treating or improving the skin of an individual. Compositions of the disclosure are particularly useful for treating skin damage related to the presence of specific strains of C. acnes. The disclosure also provides methods of using compositions for treating or improving skin damage.

One embodiment of the invention is a composition comprising one or more ingredients that alter the environment of the skin of an individual, such that the skin becomes less hospitable for the growth of undesirable strains of C. acnes, and more hospitable for the growth of desirable strains of C. acnes. The composition may comprise one or more of a prebiotic that selectively promotes the growth of desirable strains of C. acnes, a probiotic comprising one or more desirable strains of C. acnes, and a post-biotic comprising at least one molecule produced by C. acnes. The composition may promote the growth of desirable strains of C. acnes at a rate that is at least 2×, at least 3×, at least 4×, at least 5×, at least 10×, at least 50×, at least 100×, or at least 1000× greater than the growth rate of undesirable strains of C. acnes.

In one aspect, the composition comprises a prebiotic. The prebiotic may comprise a compound that selectively inhibits the growth of undesirable strains of C. acnes. The compound may inhibit the synthesis of porphyrin in undesirable strains of C. acnes. The compound may be a preferential substrate for desirable strains of C. acnes and may be a carbohydrate or non-carbohydrate moiety.

In one aspect, composition may comprise a probiotic comprising one or more desirable strains of C. acnes. The one or more desirable strains of C. acnes may be derived, obtained, or isolated from a human. The one or more desirable strains of C. acnes may be stored (e.g., cryogenically) prior to use in preparing a composition of the disclosure. The one or more desirable strains of C. acnes may produce a high level of RoxP protein. The one or more desirable strains of C. acnes may produce antimicrobial peptides (AMPs). The one or more desirable strains of C. acnes may induce the expression of high levels of AMPs in host cells, which may be epithelial cells. The one or more desirable strains of C. acnes may have similar biological profiles to C. acnes strain RT6. The one or more desirable strains of C. acnes may comprise strain RT1.

In one aspect, the composition may comprise a post-biotic that comprises at least one molecule produced by C. acnes. The post-biotic may be obtained from the supernatant of C. acnes culture, or it may be obtained from a C. acnes lysate. The post-biotic molecule may have antibacterial properties or antioxidant properties. In some embodiments, the soluble fraction may have antimicrobial activity. The post-biotic molecule may comprise a protein, a lipid, or a polysaccharide, or combinations thereof, for example a glycoprotein or a glycolipid. The post-biotic molecule may comprise a RoxP protein, which may be obtained from a culture of C. acnes, or may be produced via recombinant, enzymatic, biofermentative, or synthetic methods.

In one aspect, the composition may comprise one or more additional ingredients useful for treating skin. The additional ingredient may be a natural ingredient that may be obtained, for example, from a plant or an animal. The additional ingredient may be an active pharmaceutical ingredient (API); non-limiting examples of APIs suitable for use in compositions of the present invention include a retinoid, an extract of Cannabis species, or a concentrated or purified cannabinoid, any one or more of which may be produced by being extracted from Cannabis species, being chemically synthesized, through synthetic biology-directed fermentation, or by enzymatic biosynthesis. An additional ingredient may be a prodrug of nitric oxide (NO), a nitric oxide donor, or an extract or fraction of Camellia species rich in catechins, or a concentrated or purified catechin, extracted from Camellia species, chemically synthesized, or produced through synthetic biology-directed fermentation or enzymatic biosynthesis. An additional ingredient may be cis-resveratrol, trans-resveratrol, or a racemic or scalenic mixture thereof. An additional ingredient may include N-acetyl cysteine, sirolimus (rapamycin), pentaerythrityl tetraisostearate (PETIS), isononyl isononanoate (ININ), and PEG distearate (PDS).

In one aspect, the composition may be as a paste, a cream, a slurry, a gel, a lotion, an emollient, a spray, a rinse, or an emulsion which may be applied to the skin after it has been subjected to microneedling, also known as dermarolling. The composition may be applied after the application of a cosmetic mask preparation or cosmetic strip intended to extract resident sebaceous microbial colonies and skin lipids from the epidermis.

One aspect of the invention is a kit comprising a composition that comprises one or more ingredients that alter the environment of the skin of an individual, such that the skin becomes less hospitable for the growth of undesirable strains of C. acnes, and/or more hospitable for the growth of desirable strains of C. acnes. The composition may comprise one or more of a prebiotic of the disclosure, a probiotic of the disclosure, and a post-biotic of the disclosure. The composition may comprise a prebiotic of the disclosure, a probiotic of the disclosure, and a post-biotic of the disclosure. The kit may comprise a means for removing resident bacteria from the skin, such means including, but not limited to, pore strips, hydroactive cleaning pads, skin strips, and skin masks. The kit may comprise instructions for using the components of the kit to produce a composition of the disclosure. The kit may comprise instructions for using the composition for preventing or treating at least one of acne vulgaris and skin damage. The kit may be used in the prevention or treatment of skin damage, or in the prevention or treatment of acne vulgaris.

One embodiment of the invention is a method of treating or preventing skin damage, comprising administering to the skin of an individual a composition of the disclosure. In one aspect, resident bacteria are removed from the skin using chemical and/or mechanical means, to induce colonization with a desirable strain of C. acnes. Examples of such means include, but are not limited to, pore strips, hydroactive cleaning pads, skin strips, and skin masks.

One embodiment of the invention is a method of treating or preventing acne vulgaris, comprising administering to the skin of an individual a composition of the disclosure.

In one aspect of the present invention, a composition for improving the skin microbiome comprises at least one of (a) a prebiotic, comprising a compound that selectively promotes the growth of at least one desirable strain of Cutibacterium acnes (C. acnes); (b) a probiotic, comprising at least one desirable strain of C. acnes; and (c) a post-biotic, comprising a molecule produced by C. acnes.

In embodiments, in the presence of the compound of the prebiotic, at least one desirable strain of C. acnes may grow at a rate that is at least about two times, at least about three times, at least about four times, at least about five times, at least about ten times, at least about fifty times, at least about one hundred times, or at least about one thousand times as great as a growth rate of at least one undesirable strain of C. acnes.

In embodiments, the compound of the prebiotic may selectively inhibit the growth of at least one undesirable strain of C. acnes.

In embodiments, the compound of the prebiotic may selectively inhibit the synthesis of porphyrin in at least one undesirable strain of C. acnes.

In embodiments, the compound of the prebiotic may be a preferential substrate for at least one desirable strain of C. acnes.

In embodiments, the at least one desirable strain of C. acnes may be obtained from a human.

In embodiments, the at least one desirable strain of C. acnes may produce a high level of RoxP protein. The level of RoxP produced by the at least one desirable strain of C. acnes may, but need not, be at least about 25% greater, at least about 50% greater, at least about 75% greater, or at least about 100% greater than a level of RoxP produced by at least one undesirable strain of C. acnes.

In embodiments, the at least one desirable strain of C. acnes may produce antimicrobial peptides (AMPs).

In embodiments, the at least one desirable strain of C. acnes may induce the expression of high levels of antimicrobial peptides (AMPs) in human cells. The human cells may, but need not, be epithelial cells.

In embodiments, the at least one desirable strain of C. acnes may comprise strain RT6.

In embodiments, the post-biotic may be obtained by lysing C. acnes cells to form a lysate and removing an insoluble fraction of the lysate.

In embodiments, the molecule of the post-biotic may be effective as at least one of an antimicrobial and an antioxidant.

In embodiments, the molecule of the post-biotic may be selected from the group consisting of a protein, a lipid, and a polysaccharide.

In embodiments, the molecule of the post-biotic may be RoxP. The RoxP may, but need not, be obtained from a culture of C. acnes. The RoxP may, but need not, be produced recombinantly, enzymatically, biofermentatively, or synthetically.

In embodiments, the composition may comprise at least two of (a), (b), and (c).

In embodiments, the composition may further comprise an additional ingredient useful for treating skin. The additional ingredient may, but need not, be a naturally occurring substance. The additional ingredient may, but need not, be obtained from a plant or an animal, and may, by way of non-limiting example, comprise an extract from a Cannabis species, which may in turn comprise a catechin. The additional ingredient may, but need not, comprise at least one of a prodrug of nitric oxide and a nitric oxide donor. The additional ingredient may, but need not, comprise resveratrol, which may, by way of non-limiting example, consist essentially of either cis-resveratrol or trans-resveratrol or be a racemic or scalenic mixture of cis-resveratrol and trans-resveratrol. The additional ingredient may, but need not, comprise an active pharmaceutical ingredient (API, which may, by way of non-limiting example, be a retinoid or an inhibitor of a mammalian target of rapamycin (mTOR).

In embodiments, the composition may be formulated as a paste, a cream, a slurry, a gel, a lotion, an emollient, a spray, a rinse, a powder, or an emulsion.

In another aspect of the present invention, a kit comprises a composition of the invention as disclosed herein.

In embodiments, the kit may further comprise instructions for using the composition to prevent or treat at least one of acne vulgaris and skin damage.

In embodiments, the kit may further comprise a means for removing native bacteria from the skin. The means may, but need not, comprise at least one of a pore strip, a hydroactive cleaning pad, a skin strip, and a skin mask.

In another aspect of the present invention, a method for treating or preventing at least one of acne vulgaris and skin damage comprises administering to skin of an individual a composition of the invention as disclosed herein.

In embodiments, the skin of the individual may be infected with C. acnes.

In embodiments, the method may further comprise, prior to administration of the composition, removing native bacteria from the skin of the individual. The native bacteria may, but need not, be removed by one or more means selected from the group consisting of a pore strip, a hydroactive cleaning pad, a skin strip, and a skin mask.

These and other advantages of the present invention will be apparent from the disclosure contained herein.

For purposes of further disclosure and to comply with applicable written description and enablement requirements, the following references generally relate to Cutibacterium acnes and are hereby incorporated by reference in their entireties:
Jason M. Lazar and Douglas S. Schulman, “Propionibacterium acnes prosthetic valve endocarditis: a case of severe aortic insufficiency,” 15(4) Clinical Cardiology 299 (April 1992) (hereinafter “Lazar”).
Jian-Lin Yu et al., “Fibronectin binding by Propionibacterium acnes,” 19(3) FEMS Immunology and Medical Microbiology 247 (November 1997) (hereinafter “Yu”).
T. P. Thompson and A. Leland Albright, “Propionibacterium acnes infections of cerebrospinal fluid shunts,” 14(8) Child's Nervous System 378 (August 1998) (hereinafter “Thompson”).
Ikuo Ishige et al., “Quantitative PCR of mycobacterial and propionibacterial DNA in lymph nodes of Japanese patients with sarcoidosis,” 354(9173) The Lancet 120 (July 1999) (hereinafter “Ishige”).
W. Lloyd Clark et al., “Treatment strategies and visual acuity outcomes in chronic postoperative Propionibacterium acnes endophthalmitis,” 106(9) Ophthalmology 1665 (September 1999) (hereinafter “Clark”).
Michael M. Tunney et al., “Detection of prosthetic hip infection at revision arthroplasty by immunofluorescence microscopy and PCR amplification of the bacterial 16S rRNA gene,” 37(10) Journal of Clinical Microbiology 3281 (October 1999) (hereinafter (“Tunney”).
Jerald P. Underdahl et al., “Propionibacterium acnes as a cause of visually significant corneal ulcers,” 19(4) Cornea 451 (July 2000) (hereinafter “Underdahl”).
Abdul H. Mohsen et al., “Propionibacterium acnes endocarditis in a native valve complicated by intraventricular abscess: a case report and review,” 33(5) Scandinavian Journal of Infectious Diseases 379 (May 2001) (hereinafter “Mohsen”).
Alistair Stirling et al., “Association between sciatica and Propionibacterium acnes,” 357(9273) The Lancet 2024 (June 2001) (hereinafter “Stirling”).
Ray M. Chu et al., “Focal intracranial infections due to Propionibacterium acnes: report of three cases,” 49(3) Neurosurgery 717 (September 2001) (hereinafter “Chu”).
U.S. Patent Application Publication 2015/0086581, entitled “Fast diagnosis and personalized treatment for acne,” published 26 Mar. 2015 to Li et al. (hereinafter “Li I”).
U.S. Patent Application Publication 2017/0058328, entitled “Fast diagnosis and personalized treatment for acne,” published 2 Mar. 2017 to Li et al. (hereinafter “Li II”).
U.S. Patent Application Publication 2019/0030090, entitled “Compositions and methods for promoting skin health,” published 31 Jan. 2019 to Li et al. (hereinafter “Li III”).

As used herein, “at least one,” “one or more,” and “and/or” are open-ended expressions that are both conjunctive and disjunctive in operation. For example, each of the expressions “at least one of A, B, and C,” “at least one of A, B, or C,” one or more of A, B, and C,” “one or more of A, B, or C,” and “A, B, and/or C” means A alone, B alone, C alone, A and B together, A and C together, B and C together, or A, B, and C together.

It is to be noted that the term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The embodiments and configurations described herein are neither complete nor exhaustive. As will be appreciated, other embodiments of the invention are possible utilizing, alone or in combination, one or more of the features set forth above or described in detail below.

### DETAILED DESCRIPTION OF THE INVENTION

Unless defined otherwise, all technical and scientific terms used herein have the same meaning as is commonly understood by one of ordinary skill in the art. All patents, applications, published applications, and other publications to which reference is made herein are incorporated by reference in their entirety. In the event that there is a plurality of definitions for a term herein, the definition provided in the Summary of the Invention prevails unless otherwise stated.

The present invention is based on the finding that only certain strains of C. acnes are associated with the development of acne vulgaris. Such acne vulgaris-associated strains are considered undesirable, and elimination of such undesirable strains result in alleviation of acne vulgaris. Thus, the present invention relates to compositions, and methods of using such compositions, for treating a C. acnes infection and acne vulgaris. More specifically, the invention relates to a composition comprising agents and/or compounds for eliminating undesirable strains of C. acnes, and replacing them with desirable strains of C. acnes, as well as methods of using such a composition. Thus, a general method of the invention may be practiced by applying to a surface, such as skin, a composition that renders the surface inhospitable for undesirable strains of C. acnes, and favorable for desirable strains of C. acnes.

Before the present invention is further described, it is to be understood that this invention is not limited to the particular embodiments described, as such may, of course, vary. It is also to be understood that the terminology used herein is for the purpose of describing particular embodiments only, and is not intended to be limiting, as the scope of the present invention is limited only by the claims.

It must be noted that as used herein and in the appended claims, the singular forms “a,” “an,” and “the” include plural referents unless the context clearly dictates otherwise. For example, “a nucleic acid molecule” refers to one or more nucleic acid molecules. As such, the terms “a,” “an,” “one or more,” and “at least one” can be used interchangeably. Similarly, the terms “comprising,” “including,” and “having” can be used interchangeably. It is further noted that the claims may be drafted to exclude any optional element. As such, this statement is intended to serve as antecedent basis for use of such exclusive terminology as “solely,” “only,” and the like in connection with the recitation of claim elements or use of a “negative” limitation.

One embodiment of the invention is a composition comprising one or more ingredients selected from the group consisting of a prebiotic that selectively promotes the growth of one or more desirable strains of C. acnes, a probiotic comprising a desirable strain of C. acnes, and a post-biotic comprising at least one antioxidant protein produced by C. acnes. In one aspect, the composition may comprise one, two, or all three of these ingredients.

In one aspect, the prebiotic may selectively promote the growth of one or more desirable strains of C. acnes. As used herein, phrases such as “selectively promotes the growth of” desirable strains, “selective growth promotion of” desirable strains, and the like mean that in the presence of the prebiotic, strains deemed “desirable” will outgrow strains deemed “undesirable.” Thus, in the presence of the prebiotic, desirable strains of C. acnes will grow (i.e. replicate) at a faster rate, and/or to higher numbers, than will a strain deemed “undesirable.” In one aspect, the growth rate of a desirable strain in the presence of the prebiotic may be at least about two times, at least three times, at least four times, at least about five times, at least about ten times, at least about fifty times, at least one hundred times, or at least about one thousand times as fast as the growth rate of an undesirable strain in the presence of the prebiotic. As used herein, “growth rate” refers to the time needed for a bacterial cell to replicate, thereby producing progeny bacterial cells. In one aspect, a desirable strain may, in the presence of the prebiotic, produce at least about two times, at least about three times, at least about four times, at least about five times, at least about ten times, at least about fifty times, at least about one hundred times, or at least about one thousand times as many bacterial cells over a given period of time as may be produced by an undesirable strain in the presence of the prebiotic for the same given period of time.

In one aspect, the prebiotic may comprise a compound that selectively inhibits the growth of undesirable strains of C. acnes. The selective growth-promoting activity of the prebiotic compound may be due to direct, selective inhibition of one or more replication steps in undesirable strains of C. acnes. The prebiotic compound may completely inhibit the growth of undesirable strains of C. acnes. The prebiotic compound may be bactericidal, or it may be bacteriostatic for undesirable strains of C. acnes. In one aspect, the selective growth-promoting activity of the prebiotic may be due to selective promotion of the growth of desirable strains of C. acnes.

In one aspect, the prebiotic may comprise a compound that is a preferential substrate for desirable strains of C. acnes. In other words, the prebiotic compound may be a better, or exclusive, substrate for desirable strains of C. acnes relative to undesirable strains of C. acnes, such that in the presence of the compound, the desirable strains of C. acnes use the substrate to outgrow (e.g. grow at a faster rate than) undesirable strains of C. acnes.

The prebiotic compound may be a natural compound, or it may be a synthetic compound. As used herein, a “natural compound” is a compound that has been obtained from a product of nature. The natural compound may be isolated from the product of nature by humans. In accordance with the present invention, an “isolated” compound is one that has been removed from its natural milieu by human manipulation, and can include any compound, such as DNA, RNA, proteins, carbohydrates, lipids, or derivatives thereof. It is to be understood that the term “isolated” may not reflect the extent to which the nucleic acid molecule has been purified. Thus, a compound in a crude cell preparation (e.g. 500×g supernatant), either from plant cells, animal cells, tissue culture cells, or cells from a microorganism (e.g. bacterial cells), may be considered isolated. The term “isolated” may also refer to a compound that is substantially free of cellular material, culture medium (when produced in tissue culture), or chemical precursors or other chemicals when chemically synthesized. In one aspect, the compound is produced by chemical synthesis.

C. acnes, a Gram-positive, microaerophilic bacterium, is known to naturally produce high levels of intracellular porphyrins, a group of pro-inflammatory metabolites important in acne development. The synthesis of porphyrin begins with the production of 5-aminolevulinic acid (ALA), which can be accomplished by the condensations of glycine and succinyl CoA catalyzed by ALA synthase, or by the direct conversion of glutamate into ALA. ALA dehydratase then acts on the ALA to form monopyrrole porphobilinogen (PBG). In the next step of the pathway, four molecules of PBG are condensed to form hydroxymethylbilane, a reaction catalyzed by PBG deaminase. Hydroxymethylbilane is then converted by uroporphyrinogen (UP) III synthase to UPIII, which is converted to coproporphyrinogens (CP), in which CP III is the major normal intermediate. Decarboxylation of propionate residues in CPIII results in the production of protoporphyrinogen (PP) IX, which is then converted by PP IX oxidase to protoporphyrin IX, which is the terminal product in C. acnes. Inhibition in any one of these steps will result in failure of the cell to produce porphyrins.

As used herein, an “undesirable strain of C. acnes” is a strain that is associated with the development of acne vulgaris. In other words, undesirable strains of C. acnes are present in high numbers on the skin of individuals suffering from acne vulgaris. In contrast, undesirable strains are absent from, or present in low numbers on, the skin of individuals who do not suffer from acne vulgaris. In certain aspects, undesirable strains of C. acnes may produce low levels of RoxP protein. RoxP is a protein produced by most strains of C. acnes that has antioxidant enzymic activity (Radical oxygenase of Propionibacterium acnes), and facilitates aerobic bacterial growth in vitro and ex vivo. An example of a RoxP is represented by Genbank Accession No. AFJ11214.1. In some aspects, an undesirable strain of C. acnes may not produce any RoxP. In accordance with the present invention, a “low level” of RoxP production refers is a level (e.g. amount, concentration, enzymic activity) of RoxP produced by an undesirable stain of C. acnes relative to the level of RoxP produced by a desirable strain of C. acnes. In one aspect, the level of RoxP produced by an undesirable strain of C. acnes may be at least about 25%, at least about 50%, at least about 75%, or up to about 100% less than the level of RoxP produced by a desirable strain of C. acnes. In one aspect, the level of RoxP produced by an undesirable strain of C. acnes may be no more than about half, no more than about one third, no more than about one quarter, no more than about one fifth, no more than about one tenth, or no more than about 1% of the level of RoxP produced by a desirable strain of C. acnes. In one aspect, the undesirable strain does not produce a detectable amount of RoxP. Examples of undesirable strains of C. acnes include, but are not limited to, strain RT4, strain RT5, strain RT7, strain RT8, strain RT9, and strain RT10. Accordingly, undesirable strains of C. acnes will have biological profiles similar to these strains (e.g. their level of RoxP production will be within 20% of the level observed in these strains).

As used herein, a “desirable strain of C. acnes” is a strain that is not associated with acne vulgaris. In other words, desirable strains of C. acnes are prevalent on the skin of an individual who does not suffer from acne vulgaris. In addition, desirable strains of C. acnes produce high levels of RoxP. For example, the level of RoxP produced by a desirable strain of C. acnes may be at least about 25%, at least about 50%, at least about 75%, or at least about 100% more than the level of RoxP produced by an undesirable strain. The level of RoxP produced by a desirable strain of C. acnes produces may be at least about two times, at least about three times, at least about four times, at least about five times, at least about ten times, or at least about one hundred times more than the level of RoxP produced by an undesirable strain. One example of a desirable strain of C. acnes is strain RT6. Accordingly, desirable strains of C. acnes will have biological profiles similar to this strain (e.g. their level of RoxP production will be within 20% of the level observed in strain RT6).

In addition to being associated with healthy skin and producing high levels of RoxP, desirable strains of C. acnes may produce antimicrobial peptides (AMPs). AMPs, also called host defense peptides (HDPs), are peptides that can kill a variety of bacteria, viruses, and fungi. Moreover, desirable strains of C. acnes may induce the production of AMPs in host cells. Host cells are cells that produce compounds producing an environment sufficient for the growth of C. acnes. One example of a host cell is a human epithelial cell.

As used herein, the term “selectively inhibits” refers to a compound that preferentially inhibits the growth of one type of bacteria relative to the growth of another type of bacteria. For example, a compound that selectively inhibits the growth of an undesirable strain of C. acnes is one that has a minimal (e.g. less than 20% inhibition in cell growth numbers), or no, effect on the growth rate of cells from a desirable strain of C. acnes, but which reduces the number of progeny bacterial cells produced by cells from an undesirable strain of C. acnes. Such a compound may completely inhibit the replication of cells from an undesirable strain of C. acnes. The compound may cause cells from an undesirable strain of C. acnes to produce at least about 25% fewer, at least about 30% fewer, at least about 40% fewer, at least about 50% fewer, at least about 60% fewer, at least about 70% fewer, at least about 80% fewer, or at least about 90% fewer, cells relative to the number of cells produced in a similar culture of desirable C. acnes cells exposed to the same compound. The compound may be bacteriostatic for undesirable strains of C. acnes. The compound may be bactericidal. The compound may be bacteriostatic for undesirable strains of C. acnes. The compound may interfere with, or inhibit, a DNA polymerase, an RNA polymerase, an enzyme, cell wall synthesis, or protein production. In one aspect, the inhibitory compound may inhibit the synthesis of porphyrins in C. acnes. In one aspect, the inhibitory compound may inhibit the synthesis of porphyrins in an undesirable strain of C. acnes.

As used herein, a “preferential substrate” is a compound that can be utilized in a differential manner by two different types of bacterial cells. In the context of the present invention, a preferential substrate is one that allows desirable C. acnes cells of the invention to grow at a faster rate than undesirable C. acnes cells. The differential rate of growth may be due to differences in the uptake of the substrate by cells of the desirable and undesirable strains of C. acnes. The differential rate of growth may be due to differences in the ability of cells of the desirable and undesirable strains of C. acnes to utilize (e.g. catabolize, incorporate, etc.) the substrate. For example, a desirable strain of C. acnes may be able to take up the preferential substrate and use it to foster replication, whereas an undesirable stain of C. acnes may not be able to take up and use the substrate. Consequently, the desirable strain may replicate at a faster rate than the undesirable strain. In one aspect, undesirable strains of C. acnes are completely unable to take up or utilize the substrate.

As stated above, the probiotic may comprise a desirable strain of C. acnes. Properties of such strains have been described elsewhere herein. The desirable strain of C. acnes may be obtained from any suitable source. In one aspect, the desirable strain of C. acnes is obtained from a human. Sequencing studies of diverse skin sites of healthy adults have shown that the microbiomes of sebaceous sites comprise and are often dominated by Cutibacterium spp. The term “microbiome” refers to all of the microorganisms present in a particular community. In the context of the present disclosure, the “microbiome” refers to the community of bacteria, viruses, and fungal organisms that inhabit the epithelial surfaces. It should be appreciated that while the original sample of the desirable strain may be obtained from a human (e.g. from the skin), the desirable strain may be derived from the original sample, and may be modified, stored, and/or expanded in culture prior to use in compositions and methods of the instant disclosure. In certain aspects, the desirable strain of C. acnes is a naturally occurring strain that has been modified by directed evolution to alter one or more activities, such as the level of production of antimicrobial peptides found in low quantities in the naturally occurring strain.

As stated above, desirable strains of C. acnes produce high levels of RoxP, and are associated with an individual having healthy skin. In other words, the number of bacterial cells from a desirable strain of C. acnes is significantly greater than the number of cells from an undesirable strain of C. acnes, in individuals having healthy skin. Thus, in one aspect, the desirable strain of C. acnes used in a composition of the invention may be associated with healthy skin. The desirable strain of C. acnes used in a composition of the invention may produce a high level of RoxP. The level of RoxP produced by a desirable strain of C. acnes may be at least about 25%, at least about 50%, at least about 75%, or at least about 100% greater than the level of RoxP produced by an undesirable strain. The level of RoxP produced by a desirable strain of C. acnes may be at least about two times, at least about three times, at least about four times, at least about five times, at least about ten times, or at least about one hundred times greater than the level of RoxP produced by an undesirable strain of C. acnes. In one aspect, the desirable strains of C. acnes may induce the production of antimicrobial peptides (AMPs) in host cells. In one aspect, the desirable strain of C. acnes may have biological properties similar to strain RT6. In one aspect, the desirable strain of C. acnes may be strain RT6.

Compositions of the disclosure may also comprise a post-biotic, which may also be referred to as a post-biotic fraction or post-biotic portion. As used herein, a post-biotic comprises at least one component (e.g. protein, lipid, polysaccharide, etc.) that is produced by C. acnes. The post-biotic component may have antioxidant and/or antimicrobial activity. The post-biotic component may, but need not, be derived from a lysate of bacterial cells, such as C. acnes cells. In one aspect, the post-biotic may be obtained from the supernatant of C. acnes cultures. In one aspect, the post-biotic component may be obtained by lysing the bacterial cells and removing the insoluble fraction by low-speed centrifugation and/or filtration. In one aspect, the post-biotic component may comprise or consist of a RoxP from C. acnes. The RoxP may be obtained (e.g. purified) from the supernatant of a C. acnes culture or a lysate of C. acnes cells, or it may be produced using recombinant, enzymatic, biofermentative, or synthetic means.

One embodiment of the invention is a composition comprising a prebiotic described herein, a probiotic described herein, and a post-biotic described herein. The prebiotic may comprise a compound that selectively promotes the growth of desirable strains of C. acnes, the probiotic portion may comprise one or more desirable strains of C. acnes, and the post-biotic portion may comprise at least one molecule produced by C. acnes. The various properties of the prebiotic, probiotic, and post-biotic have been described elsewhere herein.

In one aspect, the desirable strain of C. acnes is associated with healthy skin. In one aspect, the desirable strain of C. acnes produces a high level of RoxP. In one aspect, the desirable strains of C. acnes may induce the production of antimicrobial peptides (AMPs) in host cells. In one aspect, the desirable strain of C. acnes may be strain RT6.

In addition to the ingredients described above, compositions of the disclosure may comprise additional ingredients that are beneficial for treating skin. The additional ingredient may be a natural ingredient or an artificial ingredient. A natural ingredient may be obtained from an animal, a plant, or a microorganism (e.g. bacteria, fungi, etc.). The additional ingredient may be an active pharmaceutical ingredient (API), such as a retinoid. The composition may comprise an extract of Cannabis species, or a concentrated or purified cannabinoid, such as cannabidiol and any of its diastereomers or any combination thereof, extracted from Cannabis species, chemically synthesized, or produced through synthetic biology-directed fermentation or via enzymatic biosynthesis. The composition may comprise a prodrug of nitric oxide (NO) or a nitric oxide donor. The composition may comprise an extract or fraction of Camellia species that are rich in catechins, or a concentrated or purified catechin, such as epigallocatechin gallate (EGCG). Such catechins may be extracted from Camellia species, chemically synthesized, or produced through synthetic biology-directed fermentation or enzymatic biosynthesis. The composition may comprise resveratrol, which may be cis-resveratrol, trans-resveratrol, or a racemic or scalenic mixture thereof. The resveratrol may be extracted from a plant source, chemically synthesized, or produced through synthetic biology-directed fermentation or via enzymatic biosynthesis.

Further examples of additional ingredients that may be present in compositions of the disclosure include, but are not limited to, backuchiol (e.g. (S)-bakuchiol, (R)-bakuchiol, or scalenic or racemic mixtures thereof), N-acetyl cysteine, sirolimus (rapamycin), pentaerythrityl tetraisostearate (PETIS), isononyl isononanoate (ININ), and PEG distearate (PDS).

A composition of the disclosure may be formulated for topical application. Suitable formulations include, without limitation, a cream, a slurry, a gel, a lotion, an emollient, a spray, a rinse, a powder, or an emulsion. Thus, a composition of the disclosure may comprise a moisturizer and/or a humectant. Suitable moisturizers for use in the formulations of the present invention include, but are not limited to, lactic acid and other hydroxy acids and their salts, glycerine, propylene glycol, propanediol, butylene glycol, sodium PCA, sodium hyaluronate, Carbowax 200, Carbowax 400, and Carbowax 800.

Suitable emollients or humectants for use in compositions of the disclosure include, but are not limited to, panthenol, cetyl palmitate, glycerine (glycerol), propanediol, PPG-15 stearyl ether, lanolin alcohol, lanolin, lanolin derivatives, cholesterol, petrolatum, isostearyl neopentanoate, octyl stearate, mineral oil, isocetyl stearate, myristyl myristate, octyl dodecanol, 2-ethylhexyl palmitate (octylpalmitate), dimethicone, phenyl trimethicone, cyclomethicone, C12-C15 alkyl benzoates, dimethiconol, propylene glycol, Theobroma grandiflorum seed butter, ceramides (e.g. ceramide 2 or ceramide 3), hydroxypropyl bispalmitamide MEA, hydroxypropyl bislauramide MEA, hydroxypropyl bisisostearamide MEA, 1,3-bis(N-2-(hydroxyethyl)stearoylamino)-2-hydroxy propane, bis-hydroxyethyl tocopheryl succinoylamido hydroxypropane, urea, aloe, allantoin, glycyrrhetinic acid, safflower oil, oleyl alcohol, oleic acid, stearic acid, dicaprylate/dicaprate, diethyl sebacate, isoste aryl alcohol, pentylene glycol, isononyl isononanoate, and 1,3-bis(N-2-(hydroxyethyl)palmitoylamino)-2-hydroxypropane. Appropriate combinations and mixtures of any of these moisturizing agents and emollients may be used in accordance with the present invention.

Compositions of the disclosure may also comprise components adapted to improve the stability or effectiveness of the applied formulation, such as a preservative and/or antioxidant. Suitable preservatives for use in the present invention include, but are not limited to: ureas, such as imidazolidinyl urea and diazolidinyl urea; phenoxyethanol; sodium methyl paraben, methylparaben, ethylparaben, and propylparaben; potassium sorbate; sodium benzoate; sorbic acid; benzoic acid; formaldehyde; citric acid; sodium citrate; chlorine dioxide; quaternary ammonium compounds, such as benzalkonium chloride, benzethonium chloride, cetrimide, dequalinium chloride, and cetylpyridinium chloride; mercurial agents such as phenylmercuric nitrate, phenylmercuric acetate, and thimerosal; piroctone olamine; Vitis vinifera seed oil; and alcoholic agents, for example, chlorobutanol, dichlorobenzyl alcohol, phenylethyl alcohol, and benzyl alcohol.

Suitable antioxidants include, but are not limited to, ascorbic acid and its esters, sodium bisulfate, butylated hydroxytoluene, butylated hydroxyanisole, tocopherols, tocopheryl acetate, tocopheryl succinate, sodium ascorbate/ascorbic acid, ascorbyl palmitate, propyl gallate, and chelating agents such as EDTA (e.g., disodium EDTA), citric acid, and sodium citrate.

Compositions of the invention may be administered to an individual using any suitable means. In one aspect, a composition may be applied directly to the skin of an individual. The composition may be applied to the skin after it has been subjected to microneedling, also known as dermarolling. The composition may be applied after use of a light mask, or after application of a cosmetic mask preparation or a cosmetic strip intended to extract resident sebaceous microbial colonies and skin lipids from the epidermis. In one aspect, a composition of the disclosure is applied to the skin after resident bacteria have been removed from the skin using chemical and/or mechanical means to induce colonization by a desirable strain of C. acnes. Examples of such means include, but are not limited to, pore strips, hydroactive cleaning pads, skin strips, and skin masks.

One embodiment of the invention is a kit comprising a composition of the invention, or all of the ingredients necessary for producing a composition of the disclosure. Such kits may be used to prevent skin damage, or to prevent or treat acne vulgaris in an individual. A kit of the invention may comprise a means for removing resident bacteria from the skin, such means including, but not limited to, pore strips, hydroactive cleaning pads, skin strips, and skin masks. Kits of the invention may also comprise jars, bottles, vials, applicators, and instructions for using components of the kit to produce compositions of the disclosure. Kits may also comprise instructions for using compositions of the disclosure.

One embodiment of the invention is a method of producing a composition of the disclosure, comprising combining two or more ingredients selected from the group consisting of a prebiotic that selectively promotes the growth of one or more desirable strains of C. acnes, a probiotic comprising a desirable strain of C. acnes, and a post-biotic comprising at least one antioxidant protein produced by C. acnes. In one aspect, the method comprises combining all three ingredients. The various properties of these ingredients have been described in detail elsewhere herein. In one aspect, the method may comprise one or more additional ingredients. Examples of suitable additional ingredients have been described elsewhere herein. The method may comprise adding ingredients so that the composition is in a suitable formulation for application (e.g. as a cream, a slurry, a gel, a lotion, an emollient, an emulsion, etc.). The method may also comprise adding any ingredients (e.g., antioxidants, preservatives) suitable for improving the stability and/or function of the composition. Examples of such ingredients have been described elsewhere herein.

One embodiment of the invention is a method of preventing skin damage in an individual, comprising administering to the skin of the individual a composition of the disclosure. The skin damage may or may not comprise infection with C. acnes. The individual may or may not have been diagnosed as having acne vulgaris. The individual may or may not have acne vulgaris. In one aspect, a composition of the disclosure is applied to the skin after resident bacteria have been removed from the skin using chemical and/or mechanical means to induce colonization by a desirable strain of C. acnes. Examples of such means include, but are not limited to, pore strips, hydroactive cleaning pads, skin strips, and skin masks.

One embodiment of the invention is a method of preventing or treating acne, comprising administering to the skin of an individual a composition of the disclosure. In one aspect, the individual may be at risk for developing acne. In one aspect, the individual may have acne vulgaris. The acne may or may not comprise infection with C. acnes. The individual may or may not have been diagnosed as having acne vulgaris. In one aspect, a composition of the disclosure is applied to the skin after resident bacteria have been removed from the skin using chemical and/or mechanical means to induce colonization by a desirable strain of C. acnes. Examples of such means include, but are not limited to, pore strips, hydroactive cleaning pads, skin strips, and skin masks.

The invention illustratively disclosed herein suitably may be practiced in the absence of any element which is not specifically disclosed herein. It is apparent to those skilled in the art, however, that many changes, variations, modifications, other uses, and applications of the invention are possible, and also changes, variations, modifications, other uses, and applications which do not depart from the spirit and scope of the invention are deemed to be covered by the invention, which is limited only by the claims which follow.

The foregoing discussion of the invention has been presented for purposes of illustration and description. The foregoing is not intended to limit the invention to the form or forms disclosed herein. In the foregoing Detailed Description of the Invention, for example, various features of the invention are grouped together in one or more embodiments for the purpose of streamlining the disclosure. The features of the embodiments of the invention may be combined in alternate embodiments other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description of the Invention, with each claim standing on its own as a separate preferred embodiment of the invention.

Moreover, though the description of the invention has included description of one or more embodiments and certain variations and modifications, other variations, combinations, and modifications are within the scope of the invention, e.g. as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

1. A composition for improving the skin microbiome, comprising:
(a) a prebiotic, comprising a compound that selectively promotes the growth of a first desirable strain of Cutibacterium acnes (C. acnes) and selectively inhibits the growth of, and/or the synthesis of porphyrin in, at least one undesirable strain of C. acnes;
(b) a probiotic, comprising a second desirable strain of C. acnes that produces RoxP and antimicrobial peptides (AMPs); and
(c) a post-biotic, comprising a molecule produced by C. acnes that is effective as at least one of an antimicrobial and an antioxidant,
wherein the composition is formulated for topical administration.

2. The composition of claim 1, wherein, in the presence of the compound of the prebiotic, at least one desirable strain of C. acnes grows at a rate that is at least about two times as great as a growth rate of at least one undesirable strain of C. acnes.

3. The composition of claim 1, wherein the compound of the prebiotic selectively inhibits the growth of at least one undesirable strain of C. acnes.

4. The composition of claim 1, wherein the compound of the prebiotic selectively inhibits the synthesis of porphyrin in at least one undesirable strain of C. acnes.

5. The composition of claim 1, wherein the compound of the prebiotic is a preferential substrate for at least one desirable strain of C. acnes.

6. The composition of claim 1, wherein the second desirable strain of C. acnes is obtained from a human.

7. The composition of claim 1, wherein a level of RoxP produced by the second desirable strain of C. acnes is at least about 25% greater than a level of RoxP produced by at least one undesirable strain of C. acnes.

8. The composition of claim 1, wherein the first desirable strain of C. acnes produces antimicrobial peptides (AMPs).

9. The composition of claim 1, wherein at least one of the first and second desirable strains of C. acnes induces the expression of high levels of antimicrobial peptides (AMPs) in human cells.

10. The composition of claim 9, wherein the human cells are epithelial cells.

11. The composition of claim 1, wherein at least one of the first and second desirable strains of C. acnes comprises an RT6 strain.

12. The composition of claim 1, wherein the post-biotic is obtained by lysing C. acnes cells to form a lysate and removing an insoluble fraction of the lysate.

13. The composition of claim 1, wherein the molecule of the post-biotic is selected from the group consisting of a protein, a lipid, and a polysaccharide.

14. The composition of claim 1, wherein the molecule of the post-biotic is RoxP.

15. The composition of claim 14, wherein the RoxP of the post-biotic is obtained from a culture of C. acnes.

16. The composition of claim 14, wherein the RoxP of the post-biotic is produced recombinantly, enzymatically, biofermentatively, or synthetically.

17. The composition of 1, further comprising an additional ingredient useful for treating skin.

18. The composition of claim 17, wherein the additional ingredient is a naturally occurring substance.

19. The composition of claim 17, wherein the additional ingredient is obtained from a plant or an animal.

20. The composition of claim 19, wherein the additional ingredient comprises an extract from a Cannabis species.

21. The composition of claim 20, wherein the extract comprises a catechin.

22. The composition of claim 17, wherein the additional ingredient comprises at least one of a prodrug of nitric oxide and a nitric oxide donor.

23. The composition of claim 17, wherein the additional ingredient comprises resveratrol.

24. The composition of claim 23, wherein the resveratrol consists essentially of either cis-resveratrol or trans-resveratrol.

25. The composition of claim 23, wherein the resveratrol is a racemic or scalenic mixture of cis-resveratrol and trans-resveratrol.

26. The composition of claim 17, wherein the additional ingredient comprises an active pharmaceutical ingredient (API).

27. The composition of claim 26, where the API is a retinoid.

28. The composition of claim 26, where the drug is an inhibitor of a mammalian target of rapamycin (mTOR).

29. The composition of claim 1, wherein the composition is formulated as a paste, a cream, a slurry, a gel, a lotion, an emollient, a spray, a rinse, a powder, or an emulsion.

30. The composition of claim 1, wherein the first and second desirable strains of C. acnes are the same strain.

31. The composition of claim 1, wherein the first and second desirable strains of C. acnes are different strains.

32. A kit, comprising the composition of claim 1.

33. The kit of claim 32, further comprising instructions for using the composition to prevent or treat at least one of acne vulgaris and skin damage.

34. The kit of claim 32, further comprising a means for removing native bacteria from the skin.

35. The kit of claim 34, wherein the means comprises at least one of a pore strip, a hydroactive cleaning pad, a skin strip, and a skin mask.
